(19) United States
(12) Patent Application Publication
(10) Pub. No.: US 2003/0120253 A1
(43) Pub. Date: Jun. 26, 2003

(54) Disposable absorbent article having one piece mechanical fastening system

(75) Inventors: Tom K. Wentzel, Kimberly, WI (US); Gay Lynn Wolf, Neenah, WI (US)

Correspondence Address: Senniger, Powers, Leavitt & Roedel, 16th Floor, One Metropolitan Square, St. Louis, MO, 63102, US

(73) Assignee: Kimberly-Clark Worldwide, Inc.

(21) Appl. No.: 10/032,383

(22) Filed: Dec. 21, 2001

(51) Int. Cl. A61F 13/15

(52) U.S. Cl. 604/392; 604/391

(57) ABSTRACT

The invention provides a diaper with a mechanical fastening system comprising an outer cover, at least one single piece of flexible material with an active fastening surface, a first waist region, a second waist region opposite the first waist region wherein the active fastening surface of the at least one single piece of flexible material provides an at least one fastener and the outer cover defines an inside surface and an outside surface such that in use, the active fastening surface of the at least one fastener anchors to the outside surface of the outer cover in the first waist region and the at least one fastener fastens the outside surface of the outer cover in the second waist region thereby forming a waist opening and a pair of leg openings.

### BACKGROUND OF THE INVENTION

Fastening devices have been used on clothing, disposable absorbent articles, and various miscellaneous articles such as safety belts and the like. Such devices are used when it is desirable to create a refastenable bond between two or more components or between several surfaces of the same article. In certain applications, these refastenable devices have replaced conventional buckles, zippers, buttons, snaps, tie fasteners, adhesive tape and sewing.

Disposable absorbent articles, such as diapers, generally include a liquid-permeable bodyside liner, an absorbent medium and an outer cover. In addition, disposable absorbent articles generally include some type of fastening system for fitting the article around the wearer.

Conventional disposable absorbent articles have fastening systems that typically employ multiple components such as adhesive fastening tapes, release tapes and hook material for securing the article on a wearer. Such articles typically have a fastening system wherein each fastener includes a hook material that is attached to the fastening tape. Further, each fastener typically employs a release tape that is affixed to either side of the inner liner of the article and which extends beyond the edge of the article (typically by 5 mm). The release tape attaches to the adhesive side of the fastening tape with the hook material.

The most apparent limitations of a fastening system that employs multiple components are the increased cost complexity of the fastening system relative to its functionality. Typically, the cost and functionality of these fastening systems are a tradeoff; greater functionality and performance can be achieved by using additional and more expensive materials, multiple layers of material, more complex mechanical fasteners, or a combination thereof. For example, many diaper configurations include large and complex fastener assemblies that are attached to the front and/or back side portions of the diaper for use to secure the diaper about the wearer. Manufacturing fastening assemblies with multiple layers of materials, or with sophisticated fasteners is often a complex and costly process that requires separate modules or machines in the production process in order to produce the various components necessary to construct the diaper.

Inexpensive conventional fastening systems often do not provide a reliable and functional securement of the article to the wearer. Typically, inexpensive conventional fastening systems do not accommodate the stresses imposed by fastening the article on the wearer and other stresses or displacements caused by a moving wearer while providing a desired ease of selective unfastening and removal of the article. As a result, conventional fastening systems do not offer desired levels of comfort, reliable securement, ease and low cost of manufacture and ease of use.

What is needed in the art are disposable absorbent articles (e.g., a diaper) which have a fastening system that is secure, relatively inexpensive, simple to manufacture, comfortable, and convenient to use.

### SUMMARY OF THE INVENTION

The present invention provides disposable absorbent articles (e.g., a diaper) having a mechanical fastening system that is secure, relatively inexpensive, simple to manufacture, comfortable, and convenient to use.

Specifically, the present invention provides a diaper with a mechanical fastening system comprising an outer cover, at least one single piece of flexible material with an active fastening surface, a first waist region, a second waist region opposite the first waist region wherein the active fastening surface of the at least one single piece of flexible material provides an at least one fastener and the outer cover defines an inside surface and an outside surface such that in use, the active fastening surface of the at least one fastener anchors to the outside surface of the outer cover in the first waist region and the at least one fastener fastens the outside surface of the outer cover in the second waist region thereby forming a waist opening and a pair of leg openings.

The present invention also provides a disposable absorbent article comprising an outer cover, at least one single piece of flexible material with an active fastening surface, a first waist region, a second waist region opposite the first waist region wherein the active fastening surface of the at least one single piece of flexible material provides an at least one fastener, and the outer cover defines an inside surface and an outside surface and includes a pair of ears in the first waist region such that in use, the active fastening surface of the at least one fastener anchors to the outside surface of the ears of the outer cover in the first waist region and the at least one fastener fastens the outside surface of the outer cover in the second waist region thereby forming a waist opening and a pair of leg openings.

The present invention further provides a disposable absorbent article comprising an outer cover, at least one single piece of flexible material with an active fastening surface, a first waist region, a second waist region opposite the first waist region wherein the active fastening surface of the at least one single piece of flexible material provides an at least one fastener, and the outer cover defines an inside surface and an outside surface such that in use, the active fastening surface of the at least one fastener anchors to the outside surface of the outer cover in the first waist region and is affixed to the outside surface of the outer cover in the first waist region with adhesive bonding, and the at least one fastener fastens the outside surface of the outer cover in the second waist region thereby forming a waist opening and a pair of leg openings.

### BRIEF DESCRIPTION OF THE DRAWINGS

FIG. 1 illustrates a diaper assembly of the present invention.

FIG. 2 illustrates a specific diaper of the present invention.

FIG. 3 illustrates a specific diaper of the present invention.

FIG. 4 illustrates a specific diaper of the present invention.

FIG. 5A illustrates a fastener of the present invention having a hook.

FIG. 5B illustrates a fastener of the present invention having an active fastening material comprising a plurality of hooks.

FIG. 6 illustrates a specific diaper of the present invention.

FIG. 7 illustrates a specific diaper of the present invention.

FIG. 8 illustrates a specific diaper of the present invention.

FIG. 9 illustrates a specific diaper of the present invention.

FIG. 10A illustrates an attachment panel of the present invention having a loop.

FIG. 10B illustrates an attachment panel of the present invention having an active landing material comprising a plurality of loops.

FIG. 11 illustrates a specific diaper of the present invention.

### DESCRIPTION OF PREFERRED EMBODIMENTS

In the following detailed description of the preferred embodiments, reference is made to the accompanying drawings that form a part hereof, and in which is shown by way of illustration specific embodiments in which the invention may be practiced. It is understood that other embodiments may be utilized and structural changes may be made without departing from the scope of the present invention.

The leading digit(s) of reference numbers appearing in the Figures generally correspond to the Figure number in which that component is first introduced, such that the same reference number is used throughout to refer to an identical component which appears in multiple Figures. Signals and connections may be referred to by the same reference number or label, and the actual meaning will be clear from its use in the context of the description.

The present invention described herein is directed to disposable absorbent articles (e.g., a diaper) having a mechanical fastening system wherein the active fastening material comprises a single piece of flexible material. While the present description will particularly be made in the context of a diaper article, it should be understood that the present invention is also applicable to other disposable personal care absorbent articles, such as adult incontinence articles, sanitary napkins, children's training pants and the like.

As used herein, “disposable absorbent article” refers to a disposable article which absorbs and contains body exudates. Typically, they are intended to be discarded after a limited period of use. The articles are not intended to be laundered or otherwise restored for reuse. The articles can be placed against or in proximity to the body of the wearer to absorb and contain various exudates discharged from the body.

As used herein, “chassis” refers to the body or frame of the disposable absorbent article. It will typically include one or more layers of suitable material. These layers have historically been referred to as bodyside liners, absorbent core, outer cover, etc.

As used herein, the term “layer” when used in the singular may have the dual meaning of a single element or a plurality of elements.

As used herein, the term “surface” refers to any layer, film, woven, nonwoven, laminate, composite, or the like, whether pervious or impervious to air, gas, and/or liquids.

As used herein, the term “fastener” refers to an active fastening material of the fastening system that interlocks with active landing material.

As used herein, the term “attachment panel” refers to a separate piece of active landing material of the fastening system that is affixed to the outer cover of the diaper and interlocks with at least one fastener. The active landing material may either be a separate piece of material affixed to the outer cover or the active landing material may be the cloth-like material of the outer cover itself.

As used herein, the term “hook” should be understood to encompass various geometries of protuberances that are suitable for engaging the landing zone component in order to secure the fastener. Exemplary geometries include prongs, stems, trees (such as the shapes connoted by “evergreen” and “palm” trees), mushrooms, flat tops, J-hooks, bidirectional hooks and studs protruding at various angles.

As used herein, the term “bead” refers to a continuous line of adhesive.

As used herein, the term “leg elastic member” and “waist elastic member” refers to elastic material generally adapted to fit about the legs and waist of a wearer in use to maintain a positive, contacting relationship with the wearer to effectively reduce or eliminate the leakage of body exudates from the diaper.

As used herein, the term “film” refers to a thermoplastic film made using a film extrusion and/or foaming process, such as a cast film or blown film extrusion process. For the purposes of the present invention, the term includes nonporous films as well as microporous films. Films may be vapor permeable or vapor impermeable, and function as liquid barriers under normal use conditions.

As used herein, the term “liquid permeable” refers to the ability of liquid, such as urine, to readily penetrate through the thickness of a layer or laminate under ordinary use conditions in a direction generally perpendicular to the plane of the layer or laminate at the point of liquid contact.

As used herein, the term “liquid impermeable” refers to the inability of liquid, such as urine, to readily penetrate through the thickness of a layer or laminate under ordinary use conditions in a direction generally perpendicular to the plane of the layer or laminate at the point of liquid contact.

As used herein, the term “hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved.

As used herein, the term “breathable material” refers to material which permits vapors to escape while still preventing liquid exudates from passing through.

As used herein, the term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding and hydrophobic associations.

As used herein, “thermal point bonding” refers to passing a fabric or web of fibers to be bonded between a heated calender roll and an anvil roll. The calender roll is usually, though not always, patterned in some way so that the entire fabric is not bonded across its entire surface.

As used herein, “ultrasonic bonding” refers to a process performed, for example, by passing the fabric between a sonic horn and anvil roll.

As used herein, “adhesive bonding” refers to an adhesive, such as a hot melt adhesive, that is applied between a film and a non-woven fiber material to bind the film and non-woven together. The adhesive can be applied by, for example, by melt spraying, printing or meltblowing.

As used herein, the term “flexible” refers to the capability of the fastener to flex and bend towards the attachment panel of the diaper and is further capable of flexing and bending away from the attachment panel upon disconnecting the fastener from the attachment panel.

As used herein, the term “elastic material” or “elastomeric material” refers to flexible and resilient material which tends to recover its original size and shape after removal of a force causing a deformation.

As used herein, the term “join” refers to the condition where a first member, or component, is directly or indirectly affixed, adhered, or otherwise connected to a second member or component, such as when each is directly bonded to intermediate elements..

As used herein, “affixed” or “bonded” refers to the joining, adhering, connecting, attaching, or the like, of two elements. Two elements will be considered to be bonded together when they are bonded directly to one another or indirectly to one another, such as when each is directly bonded to intermediate elements.

As used herein, the term “peel” refers to the disengagement of the fastener component from the outer cover of the diaper.

As used herein, the term “shear strength” refers to the value obtained, in grams, when subjecting joined components of the fastening device of the diaper to a disconnecting force in the direction that is generally perpendicular to the longitudinal centerline of the fastening device.

As used herein, the term “thermoplastic” refers to uncrosslinked polymers of a thermally sensitive material which flows under the application of heat or pressure.

As used herein, the term “machine direction” (MD) refers to the vertical (up-down) direction.

As used herein, the term “cross-machine direction” (CD) refers to the horizontal (across) direction.

As used herein, the term “nonwoven” and “nonwoven web” refer to fibrous materials and webs of fibrous material which are formed without the aid of a textile weaving or knitting process.

As used herein, the term “polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

As used herein, “spunbonded fibers” refers to small diameter fibers which are formed by extruding molten thermoplastic material as filaments from a plurality of fine capillaries of a spinnerette having a circular or other configuration, with the diameter of the extruded filaments then being rapidly reduced.

As used herein, “meltblown fiber” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter.

As used herein, “microfibers” refers to small diameter fibers having an average diameter not greater than about 100 microns, for example, having an average diameter of from about 0.5 microns to about 50 microns, more particularly, microfibers may have an average diameter of from about 4 microns to about 40 microns.

As used herein, “superabsorbent materials” refers to a water-swellable, water-insoluble organic or inorganic material capable, under the most favorable conditions, of absorbing at least about 15 times its weight and, more desirably at least about 30 times its weight in an aqueous solution containing 0.9 weight percent sodium chloride. The superabsorbent materials can be natural, synthetic, or a combination thereof.

As used herein, the term “skin friendly” refers to a fastening system which is substantially non-irritating and non-abrasive to human skin.

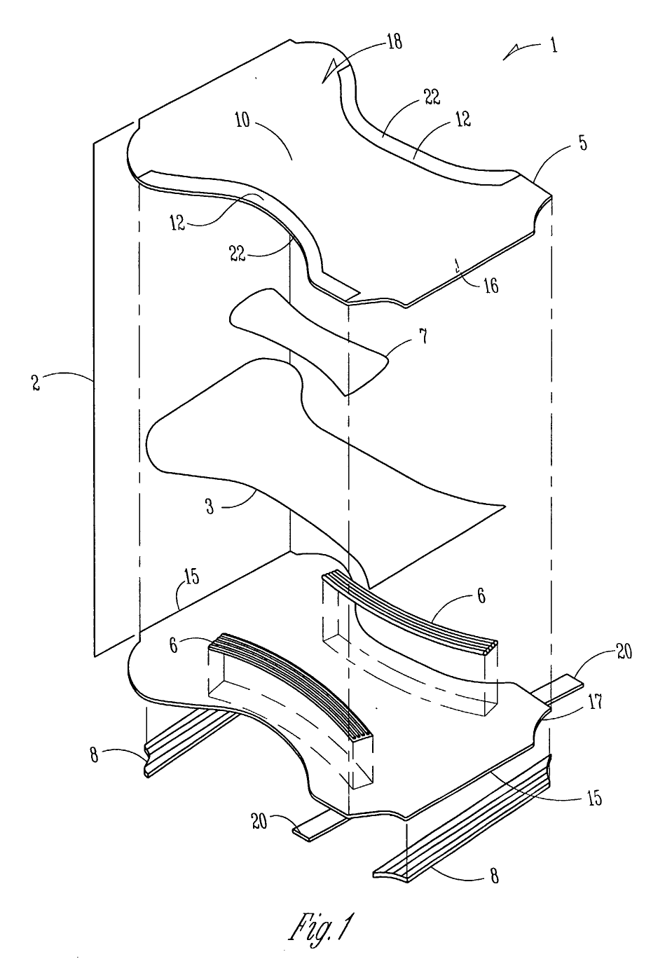

FIG. 1 is an isometric top view of a typical diaper 1 assembly. The diaper shape of diaper 1 may be of various suitable shapes. For example, in the unfastened configuration, the diaper 1 may have generally rectangular shape, T-shape, or I-shape. In the present embodiment, the diaper 1 has a generally hourglass shape in an unfastened configuration. The diaper 1 generally defines a first waist region, a second waist region and an intermediate region interconnecting the first waist region and the second waist region. Examples of diaper configurations suitable for use in connection with the instant application and other diaper components suitable for use on diapers are described in U.S. Pat. No. 4,798,603 issued Jan. 17, 1989, to Meyer et al., U.S. Pat. No. 5,176,668 issued Jan. 5, 1993, to Bemardin, U.S. Pat. No. 5,176,672 issued Jan. 5, 1993, to Bruemmer et al., U.S. Pat. No. 5,192,606 issued Mar. 9, 1993, to Proxmire et al., and U.S. Pat. No. 5,509,915 issued Apr. 23, 1996, to Hanson et al; the disclosures of which are herein incorporated by reference. The various aspects and configurations of the invention can provide distinctive combinations of softness, body conformity, reduced red-marking of the wearers skin, reduced skin hydration, improved containment of body exudates and improved aesthetics.

The various components of the diaper 1 are integrally assembled together employing various types of suitable attachment means, such as adhesive, sonic, and thermal bonds or a combination thereof. In the shown embodiment, for example, the outer cover 17 and the bodyside liner 5 are assembled to each other and to the absorbent core 3 therebetween with an adhesive, such as a hot melt, pressure-sensitive adhesive. The adhesive may be applied as a uniform continuous layer of adhesive, a patterned layer of adhesive, a sprayed pattern of adhesive, or any of separate lines, swirls or dots of adhesive. Alternatively, the absorbent core 3 may be connected to the outer cover 17 using conventional fasteners such as buttons, hook and loop type fasteners, adhesive tape fasteners, and the like. Similarly, other diaper components, such as the leg elastic members 6, the waist elastic members 8 and the fasteners 20, may be assembled into the diaper 1 article by employing the above-identified attachment mechanisms. The assembled layered components of the diaper 1 form chassis 2. Desirably, the majority of the diaper components are assembled together using ultrasonic bonding techniques for reduced manufacturing cost.

The outer cover 17 of the diaper 1, as representatively illustrated in FIG. 1, may suitably be composed of a material which is either liquid permeable or liquid impermeable. It is generally preferred that the outer cover 17 be formed from a material which is substantially impermeable to liquids. A typical outer cover 17 can be manufactured from a thin plastic film or other flexible liquid-impermeable material. For example, the outer cover 17 may be formed from a polyethylene film having a thickness of from about 0.013 millimeter (0.5 mil) to about 0.051 millimeter (2.0 mils). If it is desired to present the outer cover 17 with a more cloth-like feeling, the outer cover 17 may comprise a polyolefin film having a nonwoven web laminated to the exterior surface thereof, such as a spunbond web of polyolefin fibers. For example, a stretch-thinned polypropylene film having a thickness of about 0.015 millimeter (0.6 mil) may have thermally laminated thereto a spunbond web of polypropylene fibers. The polypropylene fibers have a thickness of about 1.5 to 2.5 denier per filament, which nonwoven web has a basis weight of about 17 grams per square meter (0.5 ounce per square yard). The outer cover 17 may otherwise include bicomponent fibers such as polyethylene/polypropylene bicomponent fibers. Methods of forming such cloth-like outer covers are known to those skilled in the art.

Further, the outer cover 17 may be formed of a woven or nonwoven fibrous web layer which has been totally or partially constructed or treated to impart a desired level of liquid impermeability to selected regions that are adjacent or proximate to the absorbent core 3. Still further, the outer cover 17 may optionally be composed of a micro-porous “breathable” material which permits vapors to escape from the absorbent core 3 while still preventing liquid exudates from passing through the outer cover 17. For example, the outer cover 17 may include a vapor permeable non-woven facing layer laminated to a micro-porous film. Suitable “breathable” outer cover materials are described in U.S. Pat. No. 5,695,868 issued to McCormack et al. and U.S. Pat. No. 5,843,056 issued Dec. 1, 1998 to Good et al., the descriptions of which are hereby incorporated by reference. Still further, the outer cover 17 may also be an elastomeric material such as a stretch-thermal laminate (STL), a neck-bonded laminate (NBL), or a stretch-bonded laminate (SBL) material. Methods of making such materials are well known to those skilled in the art and are described in U.S. Pat. No. 4,663,220 issued May 5, 1987 to Wisneski et al, and European Patent Application No. EP 0 217 032 published on Apr. 8, 1987 in the names of Taylor et al., the disclosures of which are hereby incorporated by reference. The outer cover 17 can also be embossed or otherwise provided with a matte finish to provide a more aesthetically pleasing appearance.

In the illustrated embodiments, and as described herein, the outer cover has been shown to consist substantially of a single piece of material. However, the invention also contemplates the outer cover being provided by more than one piece of material. For example the outer cover may also include a pair of ears that can extend in the lateral direction from the side edges of the diaper in one of the waist regions. In the various configurations of the invention, the ears may be integrally formed with a selected diaper component. For example, the ears can be integrally formed from the layer of material that provides the outer cover, or may be integrally formed from the material employed to provide the top surface. In alternative configurations, the ears may be provided by one or more separately provided members that are connected and assembled to the outer cover, to the top surface, in between the outer cover and top surface, or in various fixedly attached combinations of such assemblies. In certain embodiments, the fastener of the present invention may be attached to the ear portion of the outer cover.

For example, the ears may be an elastomeric material such as a neck-bonded laminate (NBL) or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference. Examples of articles that include elasticized ears and selectively configured fastener tabs are described in U.S. Pat. No. 5,496,298 issued Mar. 5, 1996 to Kuepper et al.; U.S. Pat. No. 5,540,796 to Fries; and U.S. Pat. No. 5,595,618 to Fries; the disclosures of which are also incorporated herein by reference.

The diaper 1 may further include a ventilation layer (not illustrated) located between the absorbent core 3 and the outer cover 17 to insulate the outer cover 17 from the absorbent core 3 in order to reduce the dampness of the outer cover 17 of the diaper 1.

In FIG. 1, the bodyside liner 5 suitably presents a bodyfacing surface which is compliant, soft feeling, and nonirritating to the wearers skin. Further, the bodyside liner 5 may be less hydrophilic than the absorbent core 3, to present a relatively dry surface to the wearer, and may be sufficiently porous to be liquid permeable, permitting liquid to readily penetrate through its thickness. A suitable bodyside liner 5 may be manufactured from a wide selection of web materials, such as porous foams, reticulated foams, apertured plastic films, natural fibers (for example, wood or cotton fibers), synthetic fibers (for example, polyester or polypropylene fibers), or a combination thereof. The bodyside liner 5 is suitably employed to help isolate the wearer's skin from liquids held in the absorbent core 3.

Various woven and nonwoven fabrics can be used for the bodyside liner 5. For example, the bodyside liner 5 may be composed of a meltblown or spunbonded web of polyolefin fibers. The bodyside liner 5 may also be a bonded-carded web composition of natural and/or synthetic fibers. The bodyside liner 5 may further be composed of a substantially hydrophobic material, and the hydrophobic material may optionally be treated with a surfactant or otherwise processed to impart a desired level of wettability and hydrophilicity. The bodyside liner 5 may also include nonwoven, spunbond, or polypropylene fabrics, or a combination thereof, composed of about 2.8-3.2 denier fibers formed into a web having a basis weight of about 20 grams per square meter and a density of about 0.13 grams per cubic centimeter. The fabric may be surface treated with about 0.3 weight percent of a surfactant commercially available from Hodgson Textile Chemicals, Inc. under the trade designation AHCOVEL Base N-62. The surfactant may be applied by any conventional means, such as spraying, printing, brush coating or the like. The surfactant may be applied to the entire bodyside liner 5 or may be selectively applied to particular sections of the bodyside liner 5, such as the medial section along the longitudinal centerline of the diaper, to provide greater wettability of such sections. The bodyside liner 5 may further include a composition applied thereto that is configured to be transferred to the wearer's skin for improving the skin health of the wearer. Suitable compositions for use on the bodyside liner 5 are described in U.S. Pat. No. 6,149,934 issued Nov. 21, 2000 to Krzysik et al., the disclosure of which is hereby incorporated by reference.

The absorbent core 3 of the diaper 1 may suitably include a matrix of hydrophilic fibers, such as a web of cellulosic fluff, mixed with particles of a high-absorbency material commonly known as superabsorbent material. The absorbent core 3 may include a matrix of cellulosic fluff such as wood pulp fluff and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be non-uniformly mixed. The fluff and superabsorbent particles may also be selectively placed into desired zones of the absorbent core 3 to better contain and absorb body exudates. The concentration of the superabsorbent particles may also vary through the thickness of the absorbent core 3. Alternatively, the absorbent core 3 may include a laminate of fibrous webs and superabsorbent material or other suitable means of maintaining a superabsorbent material in a localized area.

The high-absorbency material can be selected from natural, synthetic, and modified natural polymers and materials. The high absorbency materials can be inorganic materials, such as silica gels, or organic compounds, such as crosslinked polymers. Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core 3 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va., DOW DRYTECH 2035LD polymer available from Dow Chemical Co. located in Midland, Mich. and Stockhausen W65431 polymer available from Stockhausen Inc., located in Greensboro, N.C.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. As a general rule, the high absorbency material is present in the absorbent core 3 in an amount of from about 5 to about 90 weight percent based on total weight of the absorbent core 3.

The absorbent core 3 may have any of a number of shapes. For example, the absorbent core may be rectangular, I-shaped, or T-shaped. It is generally preferred that the absorbent core 3 be narrow in the crotch region 10 of the diaper 1. It has been found that the absorbent core 3 of the present invention is particularly useful when the width dimension in the crotch region 10 is from about 2.5 to about 12.7 centimeters (1.0 to about 5.0 inches), desirably no more than about 7.6 centimeters (3.0 inches) and more desirably no more than about 5.1 centimeters (2.0 inches). The narrow crotch width dimension of the absorbent core 3 allows the absorbent core 3 to better fit between the legs of the wearer. The size and the absorbent capacity of the absorbent core 3 should be compatible with the size of the intended wearer and the liquid loading imparted by the intended use of the absorbent article.

Optionally, a substantially hydrophilic tissue wrapsheet (not illustrated) may be employed to help maintain the integrity of the airlaid fibrous structure of the absorbent core 3. The tissue wrapsheet is typically placed about the absorbent core 3 over at least the two major facing surfaces thereof and composed of an absorbent cellulosic material, such as creped wadding or a high wet-strength tissue. The tissue wrapsheet can be configured to provide a wicking layer which helps to rapidly distribute liquid over the mass of absorbent fibers comprising the absorbent core 3. The wrapsheet material on one side of the absorbent fibrous mass may be bonded to the wrapsheet located on the opposite side of the fibrous mass to effectively entrap the absorbent core 3.

As representatively illustrated in FIG. 1, the disposable diaper 3 may include a pair of containment flaps 12 that are configured to provide a barrier and to contain the lateral flow of body exudates. The containment flaps 12 may be located along the laterally opposed side edges 22 of the bodyside liner 5 adjacent the side edges of the absorbent core 3. The containment flaps 12 may extend longitudinally along the entire length of the absorbent core 3 or may only extend partially along the length of the absorbent core 3. When the containment flaps 12 are shorter in length than the absorbent core 3, the containment flaps 12 can be selectively positioned anywhere along the side edges 22 of diaper 1 in the crotch region 10. In a particular aspect of the invention, the containment flaps 12 extend along the entire length of the absorbent core 3 to better contain the body exudates. Such containment flaps 12 are generally well known to those skilled in the art. For example, suitable constructions and arrangements for containment flaps 12 are described in U.S. Pat. No. 4,704,116 issued Nov. 3, 1987, to K. Enloe, the disclosure of which is hereby incorporated by reference.

The diaper 1 may include elastics such as a pair of leg elastic members 6 which are affixed to the side edges 22 of the outer cover 17 to further prevent leakage of body exudates and to support the absorbent core 3. The diaper 1 may also include a pair of waist elastic members 8 which are affixed to the longitudinally opposed waist edges 15 of the outer cover 17 or of the bodyside liner 5 (not shown) of the diaper 1. The leg elastic members 6 and the waist elastic members 8 are generally adapted to closely fit about the legs and waist of the wearer in use to maintain a positive, contacting relationship with the wearer and to effectively reduce or eliminate the leakage of body exudates from the diaper 1.

Materials suitable for use as the leg elastic members 6 and waist elastic members 8 are well known to those skilled in the art. Exemplary of such materials are sheets or strands or ribbons of a polymeric, elastomeric material which are adhered to the outer cover 17 in a stretched position, or which are attached to the outer cover 17 while the outer cover is pleated, such that elastic constrictive forces are imparted to the outer cover 17. The leg elastic members 6 may also include such materials as polyurethane, synthetic and natural rubber.

As illustrated in FIG. 1, the diaper 1 may also include a surge management layer 7 which helps to decelerate and diffuse surges or gushes of liquid that may be rapidly introduced into the absorbent core 3 of the diaper 1. Desirably, the surge management layer can rapidly accept and temporarily hold the liquid prior to releasing the liquid into the storage or retention portions of the absorbent core 3. In the illustrated embodiment, for example, the surge management layer 7 is interposed between the inwardly facing surface 16 of the bodyside liner 5 and the absorbent core 3. Alternatively, the surge management layer 7 may be located on the outwardly facing surface 18 of the bodyside liner 5. Examples of suitable surge management layers 7 are described in U.S. Pat. No. 5,486,166 entitled FIBROUS NONWOVEN WEB SURGE LAYER FOR PERSONAL CARE ABSORBENT ARTICLES AND THE LIKE by C. Ellis and D. Bishop, which issued Jan. 23, 1996 and U.S. Pat. No. 5,490,846 entitled IMPROVED SURGE MANAGEMENT FIBROUS NONWOVEN WEB FOR PERSONAL CARE ABSORBENT ARTICLES AND THE LIKE by C. Ellis and R. Everett, which issued Feb. 13, 1996; the entire disclosures of which are hereby incorporated by reference in a manner that is consistent herewith.

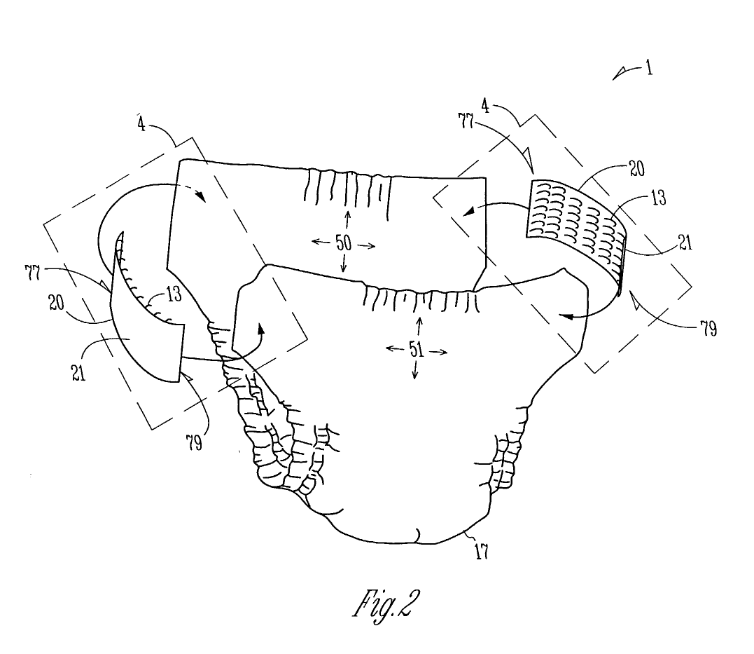

FIG. 2 is a front view of the diaper 1 and illustrates one embodiment of the mechanical fastening system 4 of the present invention in an unlocked position. In use, diaper 1 is applied to the wearer by positioning the first waist region 50, around the wearer's back and drawing the remainder of the diaper 1 (i.e., the second waist region 51) between the legs of the wearer so that the second waist region 51 of the diaper 1 is disposed across the front of the wearer and the first waist region 50 of the diaper 1 is disposed across the back of the wearer.

The invention is a simple to manufacture, cost effective mechanical fastening system 4 of the present invention. The mechanical fastening system 4 of the diaper 1 includes at least one flexible fastener 20. Specifically, the fastening system of diaper 1 may include two flexible fasteners 20 wherein each fastener 20 when fastened is located on the opposite side edges of the first waist region 50 and the second waist region 51 to create a waist opening and a pair of leg openings about the wearer.

Each fastener 20 includes active fastening material 13. The active fastening material 13 of the present invention may advantageously be hook material. The active fastening material 13 may be a separate piece of material affixed to the inside surface of the flexible material 21 which combined comprises a fastener 20, or more desirably, the active fastening material 13 is extruded from the single piece of flexible material 21 itself forming the fastener 20. If the hook material is affixed to a flexible backing, then the active fastening material 13 may be affixed to the flexible material 21 by thermal point bonding, ultrasonic bonding, adhesive bonding or a combination thereof over a portion of the width of the hook material.

The fasteners 20 may be presented, but not limited to suitable shapes which may include generally rectangle shapes, square shapes, circular shapes, triangular shapes, oval shapes, linear shapes, or a combination thereof. The fasteners 20 are preferably rectangular in shape as representatively illustrated in FIG. 2.

The preferred embodiment of the present invention is a mechanical fastening system 4 wherein the fasteners 20 anchor and fasten to the outer cover 17 wherein the outer cover 17 itself provides a “fasten anywhere” active landing material. The outer cover 17 is preferably cloth-like material, but may include loop material.

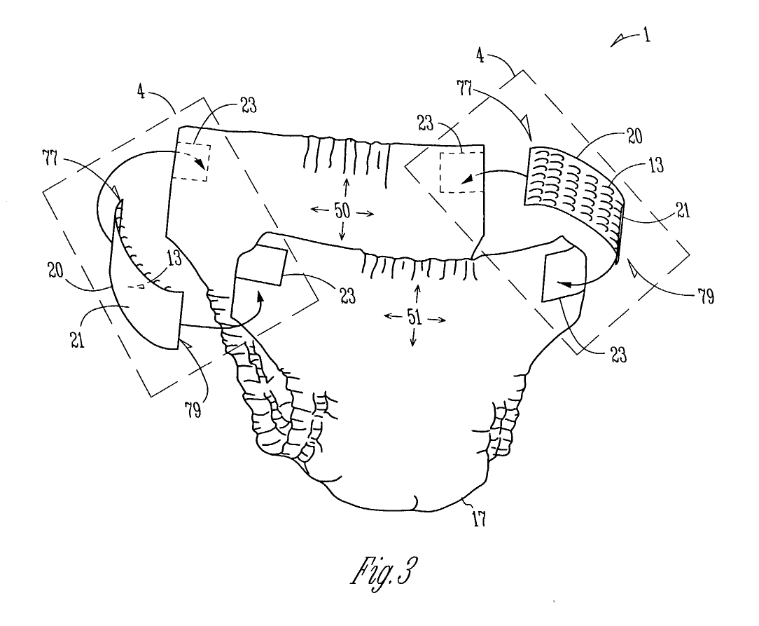

Alternatively, as representatively illustrated in FIG. 3, the diaper 1 may include one or more attachment panels 23 to which the fasteners 20 are configured to releasably engage. For example, when the fasteners 20 include hook material as illustrated, the diaper 1 may also include corresponding attachment panels 23 which may comprise complementary loop elements on the outward facing surface in the first waist region 50 and second waist region 51 of the diaper 1. The attachment panels 23 may include a woven fabric, a nonwoven fabric, a knitted fabric, a perforated or apertured layer, or the like, or a combination thereof. For example, a suitable material for the attachment panels 23 can be composed of a 2 bar, warp knit fabric of the type available from Guilford Mills, Inc., Greensboro, N.C. under the trade designation #34285, as well other of knit fabrics. (Also U.S. Pat. No. 5,858,515 to Stokes-K C patent)

The mechanical fastening system 4 is in a locked position when the anchor end 77 of each fastener 20 anchors to the first waist region 50 of the outer cover 17 and the user end 79 of each fastener 20 fastens to the second waist region 51 of the outer cover 17 to form a waist opening and a pair of leg openings about the wearer.

The preferred embodiment allows the fasteners 20 a “fasten anywhere” configuration wherein the actual anchoring points of contact of the anchor end 77 of the fasteners 20 is to the first waist region 50 of the outer cover 17 and the actual fastening points of contact of the user end 79 of the fasteners 20 is to the second waist region 51 of the outer cover 17, thereby providing the mechanical fastening system 4 in a locked position. This configuration allows the fasteners 20 the ability to conform and vary the size of the waist opening of the diaper 1 over a wide range to fit the waist of the wearer, yet provide reliable securement of the diaper 1 about the wearer. The simplicity of the mechanical fastening system 4 makes it cost effective to manufacture and assemble reducing the overall cost of the diaper 1.

Another advantage of the present invention is that the attachment points of the active fastening material of the freestanding and flexible fasteners 20 may be made at the edge of the first waist region 50 of the outer cover 17 and at the edge of the second waist region 51 of the outer cover 17, thereby reducing the required diaper circumference which, in turn, reduces the amount of material required to produce the diaper 1.

FIG. 3 is a front view of the diaper 1 and illustrates one embodiment of the mechanical fastening system 4 in an unlocked position. The present embodiment includes two separate flexible fasteners 20 that are generally rectangular in shape.

The present embodiment illustrates four flexible generally square pieces of active landing material affixed to the outer cover 17 of the diaper 1. The attachment panels 23 may be presented, but not limited to suitable shapes which may include rectangle shapes, square shapes, circular shapes, triangular shapes, oval shapes, linear shapes, and the like, or a combination thereof. The four attachment panels 23 are preferably cloth-like, but may include loop material, or other materials. As representatively illustrated, the attachment panels 23 have a generally square shape. Two attachment panels 23 are affixed to the first waist region 50 of the outer cover 17 and the other two attachment panels 23 are affixed to the second waist region 51 of the outer cover 17. The attachment panels 23 may be affixed to the outer cover 17 by thermal point bonding, ultrasonic bonding, adhesive bonding or a combination thereof over a portion of the width of the hook material.

The anchor end 77 of each fastener 20 anchors to a attachment panel 23 affixed to the first waist region 50 of the outer cover 17. The mechanical fastening system 4 is in a locked position when the user end 79 of each fastener 20 fastens to a attachment panel 23 affixed to the second waist region 51 of the outer cover 17 to form a waist opening and a pair of leg openings about the wearer.

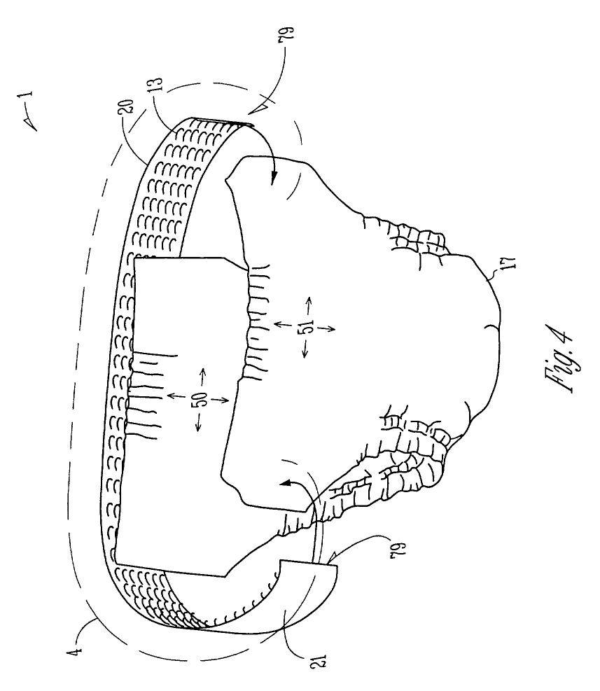

FIG. 4 is a front view of one embodiment of the diaper 1 in an unlocked configuration. In this embodiment, the mechanical fastening system 4 includes a single fastener 20 that is one elongated flexible piece of generally rectangular material that wraps around the entire first waist region 50. The fastener 20 wraps around and anchors to the active landing material of the first waist region 50 of the outer cover 17. The two user ends 79 of the fastener 20 fasten anywhere on the active landing material of the second waist region 51 of the outer cover 17.

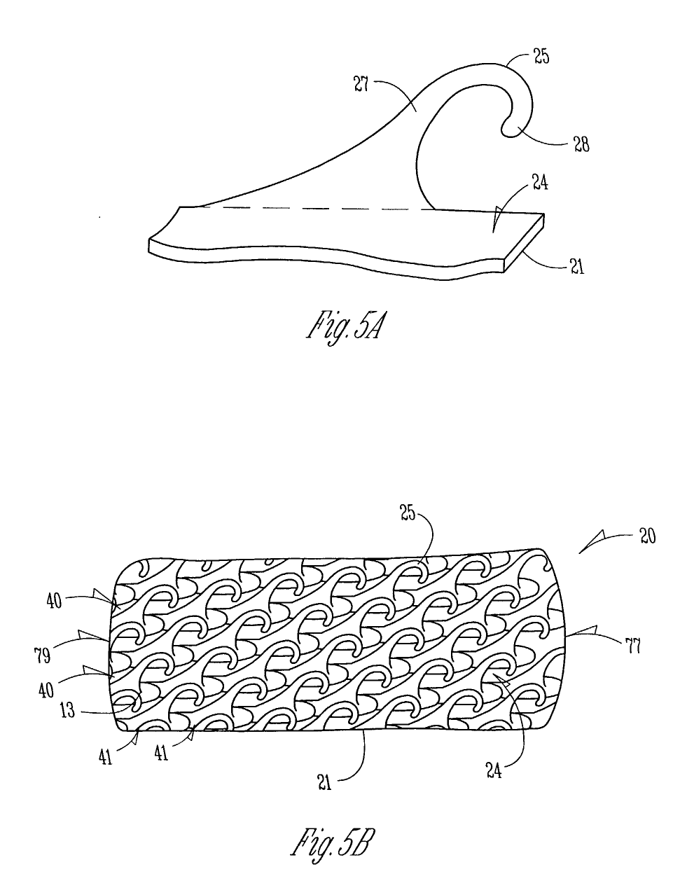

FIG. 5A illustrates a side view of one embodiment that is exemplary of the hooked material that may be utilized in the fasteners 20 of the mechanical fastening system 4. An outward projecting single hook 25 configuration (a plurality of which forms active fastening material 13 as illustrated in FIG. 3B) is preferably extruded from the single piece of flexible material 21 itself. Alternatively, the active fastening material 13 may be incorporated into the inside surface 24 of the single piece of flexible material 21 forming a fastener 20. The hook 25 includes a head 28 disposed on top of the stem or shank 27. Hooks 25 are extruded from or incorporated into a single continuous piece of flexible material (i.e., hooked material). Each fastener 20 includes a plurality of hook 25 material forming an active fastening material 13. If the hooked material is affixed to a flexible backing, then the active fastening material 13 may be affixed to the flexible material 21 by thermal point bonding, ultrasonic bonding, adhesive bonding or a combination thereof over a portion of the width of the hook material.

FIG. 5B illustrates a top view of one embodiment of the inside surface 21 of a single fastener 20. A plurality of hooks 25 form the active fastening material 13 of the shown fastener 20. The active fastening material 13 may be of various patterns and densities to achieve the desired shear strength required for the particular application of the diaper 1. In the present invention, it is desired that the anchor ends 77 of the fasteners 20 each have a shear strength of greater than 3,000 grams tensile. In order to achieve the desired strength, this may require that the hook material on the anchor ends 77 of the fasteners 20 each have a length of about 0.75 to about 1.25 inches and a width of about 1.5 to about 4.0 inches depending on the efficiency of engagement and strength of the outer cover 17. It is also desired that the user end 79 of the fasteners 20 each have a shear strength of greater than 2,000 grams tensile. In order to achieve the desired strength, this may require that the user end of the fasteners 20 each have a width of about 0.75 to about 2.0 inches.

For example, the anchor end 77 of each fastener 20 may have a length of 1.00 inch and a width of 3 inches and the user end 79 of each fastener 20 may have a length of 0.75 inch and a width of 1.25 inches. The surface area of active fastening material of the anchor end 77 of the fastener 20 would then have a surface area of 3 inches squared whereas the surface area of active fastening material of the user end 79 may be approximately 1 inch squared, or approximately one third the surface area of the anchor end 77 of the fastener 20. The size of the fastener 20 may vary in size from the anchor end 77 to the user end 79 to accommodate the desired shear strengths from of the opposing fastener 20 ends. A greater surface area of active fastening material on the anchor end 77 of the fastener 20 is generally desirable. For example, as the diaper 1 is applied to the wearer, the caregiver may attach the anchor end 77 of the fasteners 20 to the first waist region 50 to better fit the diaper 1 around the waist and buttocks of the wearer and pull the user end 79 of the fasteners 20 toward the second waist region 51 to fasten the user end 79 of the fasteners 20 to second waist region 51 of the outer cover 17. The pulling of the anchor end 77 of the fasteners 20 as the caregiver fastens the first waist region 50 to the second waist region 51 and the natural stretching of the first waist region 50 about the buttocks of the wearer may create greater stress tensions to the anchor end 77 of the fasteners 20. A greater surface area of active fastening material on the anchor end 77 of the fasteners 20 relative to the user end 79 of the fasteners 20 may provide the necessary shear strength of greater than 3,000 grams tensile on the anchor end 77 side of the fasteners 20. Desirably, the anchor end 79 of the fasteners 20 is attached to the diaper 1 prior to packaging.

Various numbers, sizes, shapes, compositions, densities, or a combination thereof of hooks 25 may be utilized in a particilar fastener 20 to provide the desired comfort and shear strengths between the anchor end 77 of the fastener 20 and the user end 79 of the fastener 20. For example, the active fastening material 13 of a fastener 20 may encompass various geometries of protuberances that are suitable for engaging the landing zone components 23. Exemplary geometries include prongs, stems, trees (such as the shapes connoted by “evergreen” and “palm” trees), mushrooms, flat tops, J-hooks, bi-directional hooks, studs, or a combination thereof protruding at various angles. For example, a flat top hook 25 material advantageously presents a surface that is less likely to expose the wearer to any coarse, sharp edges and provides a more smooth-feeling fastener surface. As such, the flat top hook 25 material provides a fastener 20 that may reduce the possibility of skin irritation and discomfort to the wearer and/or the caregiver. In addition, the flat top hook 25 material advantageously provides reliable anchorage to the first waist region 50 of the outer cover 17 of the diaper 1.

Skin friendly fasteners 20 typically have a active fastening material 13 of about 1 row of hooks/cm² to about 10 rows of hooks/cm² (about 2.5 to about 25 rows per square inch) independently in the machine direction 41, the cross-machine direction 40, or a combination thereof. Specifically, a active fastening material 13 with about 4 rows of hooks/cm² to about 30 rows of hooks/cm² (about 10 to about 50 rows per square inch) independently in the machine direction 41, the cross-machine direction 40, or a combination thereof, may be substantially non-irritating and non-abrasive to human-skin.

The number of hooks 25 in each row of the active fastening material 13 should not be so closely spaced as to interfere with and prevent the fastener 20 from effectively fastening to the outer cover 17 material or the attachment panel 23 of the outer cover 17. Conversely, the active fastening material 13 should not be so distantly spaced as to require an excessive surface area of material on the anchor side and on the user side of the fastener 20 to achieve the desired shear strength. The active fastening material 13 density may also be described in terms of the number of hooks 25 per square centimeter. It is possible to fabricate hooked material having a hook 25 density of from about 60 hooks/cm² to about 1600 hooks/cm². More desirably, the hooked material has a hook 25 density of from about 100 hooks/cm² to about 750 hooks/cm².

The present invention may also utilize selective areas of active fastening material on the inside surface 24 of a particular fastener 20. These selected areas may be referred to as “islands” wherein each fastener 20 includes a plurality of islands preferably comprised of hook 25 material. The active fastening material of each island of a fastener 20 is desirably extruded from at least two specific areas of the single piece of flexible material 21 itself or the active fastening material of each island of a fastener 20 may include at least two separate active fastening materials incorporated into the inside surface 24 of the single piece of flexible material 21. The fastener 20 includes at least one island of active fastening material on the anchor end 77 of the fastener 20 and the fastener 20 includes at least one island of active fastening material on the user end 79 of the fastener 20. The islands may be presented, but not limited to suitable shapes which may include rectangle shapes, square shapes, circular shapes, triangular shapes, oval shapes, linear shapes, or a combination thereof.

Each island of active fastening material desirably includes a hook 25 density of about 2 cm² or less, of about 2 cm² to about 8 cm², of about 8 cm² or more, or a combination thereof. A substantially low hook density of about 2 cm² or less allows the fastener 20 to have a lower manufacturing cost and a higher user skin friendliness, but a lower shear strength. Increasing the hook area to a substantially moderate hook density of about 2 cm² to about 8 cm² increases the cost of manufacture, but desirably improves the anchoring/fastening capability of the fastener 20 while maintaining user skin friendliness.

The active fastening material 13 may be manufactured from any suitable material. The active fastening material 13 is preferentially made of thermoplastic material. Hot melt adhesive thermoplastics are particularly well suited to manufacture hooks 25 of the present invention.

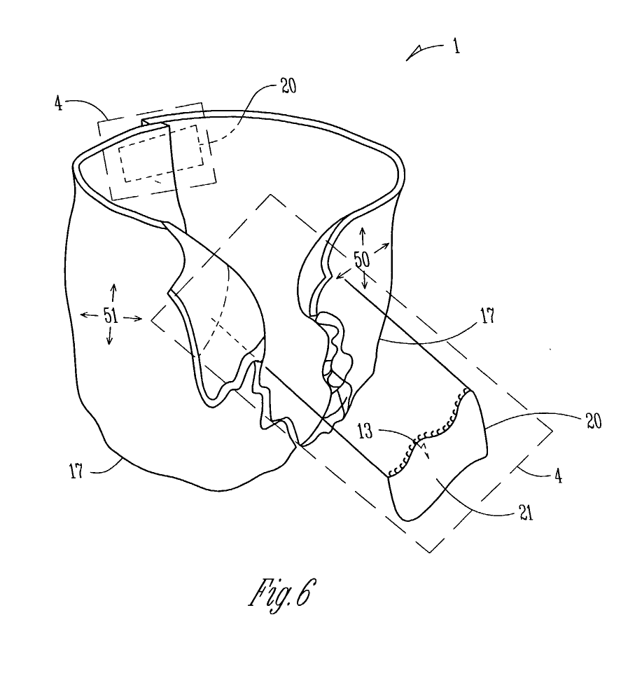

FIG. 6 illustrates a side view of one embodiment of the mechanical fastening system 4 of the diaper 1 wherein one fastener 20 is in a locked position and one fastener 20 is in an unlocked position. The locked position of diaper 1 is achieved by aligning the first waist region 50 and the second waist region 51 such that the fastener 20 is able to align and anchor to the first waist region 50 of the outer cover 17 and align and fasten to the second waist region 51 of the outer cover 17. The active landing material of the outer cover 17 allows the locked fastener 20 to anchor anywhere on the first waist region 50 of the outer cover 17 and fasten anywhere on the second waist region 51 of the outer cover 17.

FIG. 6 also illustrates one fastener 20 in an unlocked position with the active fastening material 13 affixed to the inside surface 24 of the fastener 20 that is unanchored to the first waist region 50 of the outer cover 17 and unfastened to the second waist region 51 of the outer cover 17.

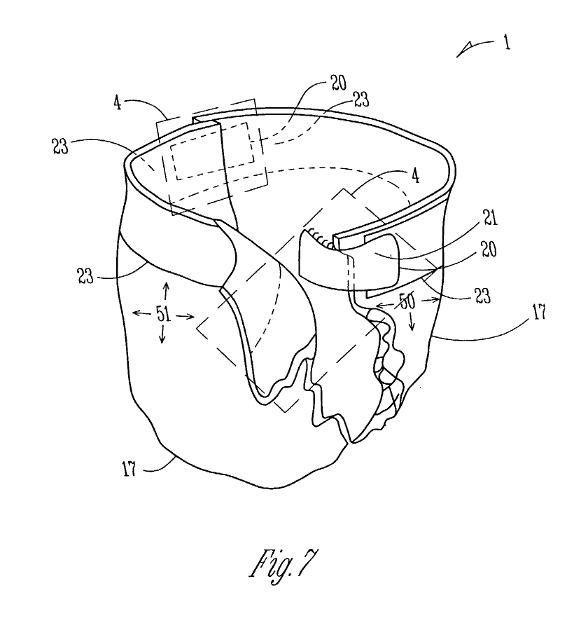

FIG. 7 illustrates a side view of one embodiment of the mechanical fastening system 4 of the diaper 1 wherein one fastener 20 is in a locked position and one fastener 20 is in an unlocked position. The present embodiment of the diaper 1 also utilizes two attachment panels 23 with generally rectangular shapes wherein one attachment panel is affixed to the first waist region 50 of the outer cover 17 and wherein one attachment panel is affixed to the second waist region 51 of the outer cover 17.

The locked position is achieved by aligning the first waist region 50 and the second waist region 51 such that the fastener 20 is able to align and anchor to the attachment panel 23 affixed on the first waist region 50 of the outer cover 17 and align and fasten to the attachment panel 23 affixed on the front portion 51 of the outer cover 17. The one unlocked fastener 20 is anchored to the attachment panel 23 of the first waist region 50 of the outer cover 17, but unfastened to the attachment panel 23 of the second waist region 51 of the outer cover 17.

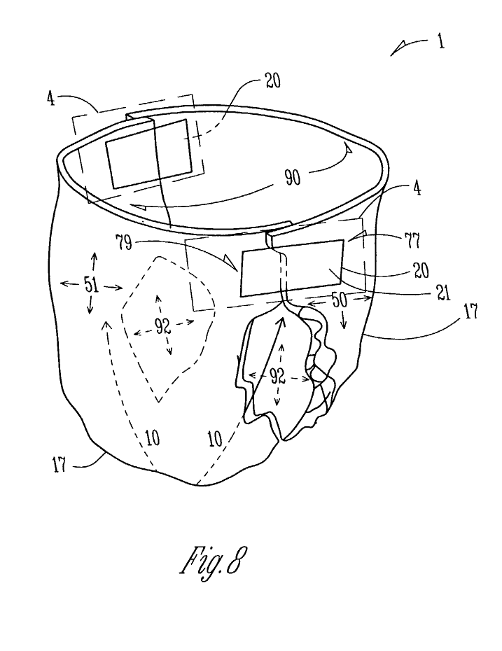

FIG. 8 is a side view of one embodiment of the diaper 1 and illustrates one embodiment of the mechanical fastening system 4 of the diaper 1 wherein both fasteners 20 are in a locked or fastened position. The mechanical fastening system 4 is in a locked position as the first waist region 50 is aligned with the second waist region 51 such that the fasteners 20 align and anchor to the first waist region 50 of the outer cover 17 and align and fasten to the second waist region 51 of the outer cover 17.

FIG. 8 illustrates the diaper 1 in a fastened position wherein the crotch region 10, the first waist region 50 and the second waist 51 together define a three-dimensional diaper 1 configuration having a waist opening 90 and a pair of leg openings 92. The first waist region 50 and the second waist region 51 of the diaper 1 are configured to encircle the waist of the wearer when worn and provide the waist opening 90 which defines a waist perimeter dimension of the diaper 1. Portions of the transversely opposed side edges of the crotch region 10 generally define the leg openings 92. The second waist region 51 includes the portion of the diaper 1 which, when worn, is positioned on the front of the wearer while the first waist region 50 includes the portion of the diaper which, when worn, is positioned on the back of the wearer. The crotch region 10 of the diaper 1 includes the portion of the diaper 1 which, when worn, is positioned between the legs of the wearer and covers the lower torso of the wearer.

One advantage of the freestanding and flexible hooked fasteners 20 is that in certain embodiments, the attachment points of the fasteners 20 to the outer cover 17 may be anchored to the edge of the first waist region 50 and/or fastened to the edge of the second waist region 51, thereby reducing the required diaper circumference which, in turn, reduces the amount of material required to produce the diaper 1.

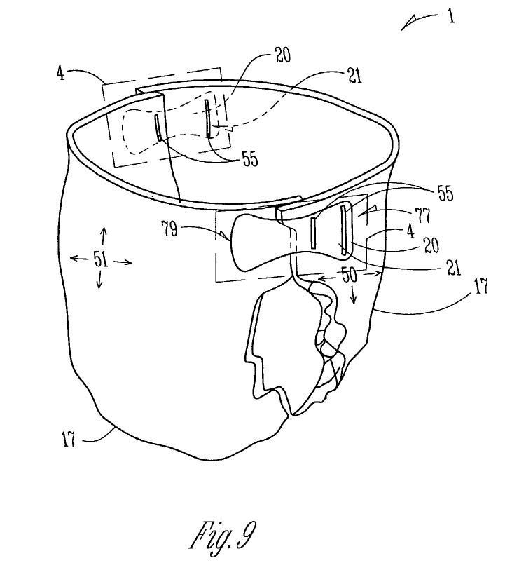

FIG. 9 is a side view of one embodiment of the mechanical fastening system 4 of the diaper 1 wherein both fasteners 20 are in a locked or fastened position. As described in FIG. 8, the mechanical fastening system 4 of the present embodiment is in a locked position as the first waist region 50 is aligned with the second waist region 51 such that the fasteners 20 align and anchor to the first waist region 50 of the outer cover 17 and align and fasten to the second waist region 51 of the outer cover 17.

As indicated above, it is desired in the present invention that the anchor end of the fasteners 20 each have a shear strength of greater than 3,000 grams tensile. In order to achieve the desired strength, this may require that the hooked material on the anchor end of the fasteners 20 each have a length of about 0.75 to about 1.25 inches and a width of about 1.5 to about 4.0 inches.

In order to maintain a uniform sheer strength of 3,000 grams tensile throughout the entire surface area of the anchor end of the fasteners 20 and to avoid peeling of the anchor end of the fasteners 20, the anchor end of the fasteners 20 may be augmented with adhesives, bonding, a larger surface area of the anchor end of the fasteners 20, various sizes, shapes, densities, and/or composition of hooks 25 on the anchor end of the fasteners 20, or a combination thereof, for more aggressive securement of the anchor end of the fasteners 20 to the outer cover 17.

As also indicated above, it is desired that the user end of the fasteners 20 each have a shear strength of greater than 2,000 grams tensile. In order to achieve the desired strength, this may require that the user end of the fasteners 20 each have a width of about 0.75 to about 2.0 inches.

In order to maintain a uniform sheer strength of 2,000 grams tensile throughout the entire surface area of the user end of the fasteners 20, and to avoid peeling of the user end of the fasteners 20, the user end of the fasteners 20 may be augmented with a larger surface area, various sizes, shapes, densities, and/or composition of hooks 25, or a combination thereof, for more aggressive securement of the user end of the fasteners 20 to the outer cover 17.

The above methods of improving the anchoring/fastening performance of the fasteners 20 on both the anchor end and the user end keeps the fasteners 20 flush against the outer cover 17 and inhibits peeling of the fasteners 20 from the outer cover 17. Peeling of the fasteners 20 is undesirable as exposed hooks 25 may cause skin irritation or abrasion to the user and/or wearer.

With respect to adhesives, FIG. 9 illustrates at least one adhesive bead 55 that may anchor the anchor end 77 of the fasteners 20 to the first waist region 50 of the outer cover 17. The anchor end 77 of the fasteners 20 are ideally augmented with two adhesive beads 55 on the inside surface 21 of each of the fasteners 20: one adhesive bead 55 on the outer most edge of the active fastening material 13 on the anchor end 77 and one adhesive bead 55 on the active fastening material 13 distal to the one adhesive bead 55 on the outer most edge of the active fastening material 13 that aligns with the outside edge of the first waist region 50 of the outer cover 17. Desirably, each adhesive bead 55 is less than 0.25 inches in width.

In an alternative embodiment, the second waist region 51 of the outer cover 17 of the diaper 1 may be the anchor end 77 and the first waist region 50 of the outer cover 17 of the diaper 1 may be the user side. Accordingly, the at least one adhesive bead 55 anchors the anchor end 77 of the fasteners 20 to the second waist region 51 of the outer cover 17. The anchor end of the fasteners 20 are ideally augmented with two adhesive beads 55 on the inside surface 24 of each of the fasteners 20: one adhesive bead 55 on the outer most edge of the active fastening material 13 on the anchor end 77 and one adhesive bead 55 on the active fastening material 13 distal to the one adhesive bead 55 on the outer most edge of the active fastening material 13 that aligns with the outside edge of the second waist region 51 of the outer cover 17. Desirably each adhesive bead 55 is less than 0.25 inches in width.

Alternatively the adhesive bead 55 may substituted by any pattern of separate lines, swirls, dots, or combination thereof in the horizontal and/or vertical direction.

The shear strength of the fasteners 20 may be further augmented by bonding the anchor end of the fasteners 21 to the first waist region 50 of the outer cover 17, or in an alternative embodiment, to the second waist region 51 of the outer cover 17. Bonding methods may include thermal point bonding, ultrasonic bonding, adhesive bonding, or a combination thereof.

The shear strength of the fasteners 20 may be increased by proportionally increasing the material size of the fasteners 20 on the anchor end and/or on the user end which in turn would proportionately increase the surface area of the active fastening material 13 on the inside surface 21 of the fasteners 20. A greater surface area of the active fastening material will increase the gripping capability of the fasteners 20 to the first waist region 50 of outer cover 17 and/or to the second waist region 51 of the outer cover 17. Further, the inside surface 21 of the fasteners 20 may embody various sizes, shapes, densities and/or compositions of hooks 25 on the anchor side to create a more aggressive fastener 20 with increased shear strength.

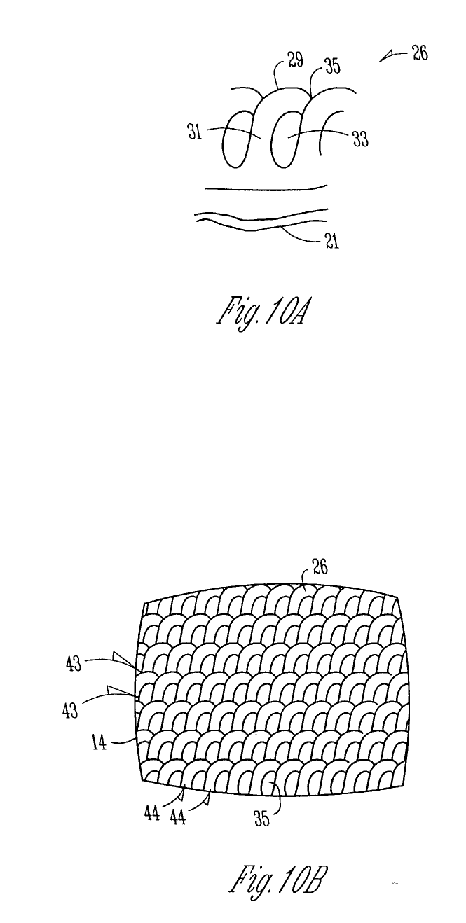

FIG. 10A is an illustrative side view of one embodiment of an example of the loop material that may be provided by the outer cover 17 itself, or utilized in a single attachment panel 23. In this embodiment, the single loop 26 (a plurality of which forms the active landing material 14 as shown in FIG. 10B) includes the shank 31 and the head 29 wherein the distal end 35 blends into an adjacent shank. The active landing material 14 is incorporated into specified attachment panels 23, or more desirably, provided by the outer cover 17 itself. The shank 31 and the head 29 project outwardly from the attachment panel 23 in an arcuate manner. Each head 29 of each loop 26 terminates at a distal end 35 to an adjacent shank 31 of another loop to form the receiving opening 33. The arcuate projection of the shank 31 and the head 29 allow the distal end 35 to contact and join one or more adjacent shanks 31 as shown, or in some embodiments, to fuse with itself. In either case, the receiving opening 33 of the loop 26 is formed to receive the head 28 and the shank 27 of the hook 25 (See FIG. 5A). When the attachment panels 23 or outer cover 17 receive and interlock with the fasteners 20, the mechanical fastening system 4 is in a locked or fastened position.

FIG. 10B is a top view of one embodiment of a portion of the outer cover 17 material or an attachment panel 23. In this embodiment, the outer cover 17 or attachment panel 23 includes a plurality of loops 26 forming the active landing material 14. The active landing material 14 may also be of various patterns and densities to achieve the desired shear strength required for the particular application of the diaper 1.

The active landing material 14 should not be so closely spaced as to interfere with and prevent the hooks 25 of the active fastening material 13 from effectively mating with the receiving openings 35 of the loops 26 of the active landing material 14. Conversely, the active landing material 14 should not be so sparse as to require a substantial increase in the size of the fastening zone member 23, excessive material of the user end of the fastener 20, or a combination thereof.

It is further desirable that if loops 26 are utilized on the outer cover 17 or on the attachment panel 23, the loop configuration should be skin friendly. Typically, the active landing material 14 includes about 2 rows to about 20 rows of loops/cm² (about 5 to about 50 rows per square inch) independently in the machine direction 44, the cross-machine direction 43, or a combination thereof. Specifically, an active landing material 14 with about 8 rows to about 40 rows of loops/cm² (about 20 rows to 100 rows per square inch) independently in the machine direction 44, the cross machine direction 43, or a combination thereof is substantially non-irritating and non-abrasive to human skin.

With respect to FIGS. 5B and 10B, it is advantageous to dispose of the active fastening material 13 and the active landing material 14 in rows, so that each hook 25 and each loop 26 are generally equally spaced from adjacent hooks and loops. Rows are generally oriented in the machine direction 41, 44 and in the cross-machine direction 40, 43. Generally, each machine direction row 41 and cross-machine direction row 40 of the active fastening material 13 and each machine direction row 44 and cross-machine direction row 43 of the active landing material 14 should be substantially and essentially equidistantly spaced from the adjacent machine direction and cross-machine direction rows of hook and loops to provide a generally uniform stress field throughout the fastener 20 and the attachment panel 23 when in a locked position, or when separation forces are applied in order to create an unlocked position of the mechanical fastening system 4.

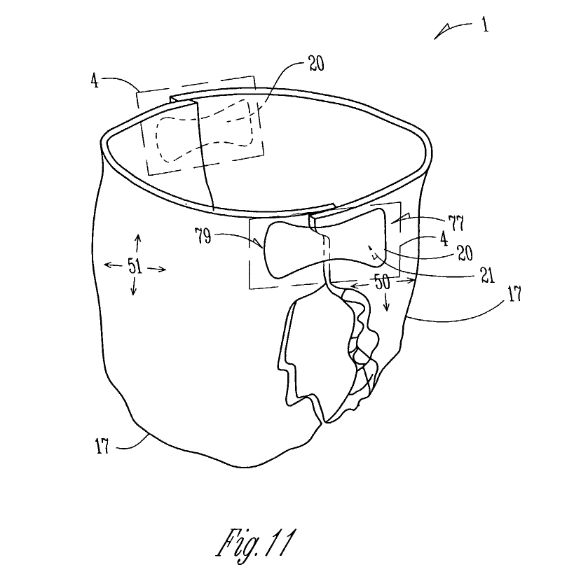

FIG. 11 is a side view of one embodiment of the mechanical fastening system 4 of the diaper 1 in a locked position. The fasteners 20 are anchored to the first waist region 50 of the outer cover 17 and fastened to the second waist region 51 of the outer cover 17. The preferred embodiment allows the fasteners 20 a “fasten anywhere” configuration wherein the actual anchoring points of the anchor end 77 of the fasteners 20 to the first waist region 50 of the outer cover 17 and the actual fastening points of the user end 79 of the fasteners 20 to the second waist region 51 of the outer cover 17 comprises the mechanical fastening system 4 in a locked position. This configuration allows the fasteners 20 the ability to conform and vary the size of the waist opening of the diaper 1 over a wide range to fit the waist of the wearer, yet provide reliable securement of the diaper 1 about the wearer. The simplicity of the mechanical fastening system 4 of the present embodiment makes it cost effective to manufacture and assemble reducing the overall cost of the diaper 1.

Methods of manufacturing the disposable absorbent article of the present invention, methods of manufacturing individual components useful to make the disposable absorbent article of the present invention, as well as methods of using the disposable absorbent article of the present invention are disclosed, e.g., in U.S. Pat. Nos. 6,296,629; 6,210,389; 6,149,934; 5,843,056; 5,695,868; 5,595,618; 5,586,371; 5,540,796; 5,509,915; 5,496,298; 5,490,846; 5,486,166; 5,336,552; 5,226,992; 5,192,606; 5,176,672; 4,704,116; 5,176,668; 4,798,603; 4,663,220; U.S. application Ser. No. 09/899,317; European Patent Application No. EP 0 217 032 and references cited therein.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled. All patents, patent documents, and references cited and disclosed herein are incorporated by reference herein.

What is claimed is:

1. A disposable absorbent article comprising:
(i) an outer cover
(ii) at least one single piece of flexible material with an active fastening surface;
(iii) a first waist region;
(iv) a second waist region opposite the first waist region wherein:
(a) the active fastening surface of the at least one single piece of flexible material provides an at least one fastener; and
(b) the outer cover defines an inside surface and an outside surface; such that in use, the active fastening surface of the at least one fastener anchors to the outside surface of the outer cover in the first waist region and the at least one fastener fastens the outside surface of the outer cover in the second waist region thereby forming a waist opening and a pair of leg openings.

2. The disposable absorbent article of claim 1, which is an incontinence product.

3. The disposable absorbent article of claim 1, which is a diaper.

4. The disposable absorbent article of claim 1, wherein the at least one fastener comprises a generally rectangular shape, square shape, circular shape, triangular shape, oval shape, linear shape, or a combination thereof.

5. The disposable absorbent article of claim 1, wherein the at least one fastener is generally rectangular in shape.

6. The disposable absorbent article of claim 1, wherein the active fastening surface of the at least one single piece of flexible material is substantially covered by active fastening material.

7. The disposable absorbent article of claim 6, wherein the active fastening material comprises a plurality of hooks.

8. The disposable absorbent article of claim 7, wherein the plurality of hooks are configured in rows of about 4 rows of hooks/cm² to about 30 rows of hooks/cm² independently in the machine direction, the cross-machine, or a combination thereof.

9. The disposable absorbent article of claim 7, wherein the plurality of hooks have a density of about 100 hooks/cm² to about 750 hooks/cm² independently in the machine direction, cross machine direction, or a combination thereof.

10. The disposable absorbent article of claim 7, wherein the active fastening material is configured in at least two selective surface areas; wherein each selective surface area has a hook density of about 2 cm² to about 8 cm² independently in the machine direction, cross machine direction, or a combination thereof.

11. The disposable absorbent article of claim 1, wherein the at least one fastener comprises a anchor end and a user end.

12. The disposable absorbent article of claim 11, wherein the anchor end, the user end, or both of the at least one fastener comprises a generally rectangular shape, square shape, circular shape, triangular shape, oval shape, linear shape, or a combination thereof.

13. The disposable absorbent article of claim 11, wherein the anchor end of the at least one fastener has a shear strength of greater than 3,000 grams tensile.

14. The disposable absorbent article of claim 11, wherein the anchor end of the at least one fastener has a length of about 0.75 to about 1.25 inches and a width of about 1.5 to about 4.0 inches.

15. The disposable absorbent article of claim 11, wherein the user end of the at least one fastener has a shear strength of greater than 2,000 grams tensile.

16. The disposable absorbent article of claim 11, wherein the user end of the at least one fastener has a width of about 0.75 to about 2.0 inches.

17. The disposable absorbent article of claim 11, wherein the anchor end of the at least one fastener is affixed to the at least one outside surface of the outer cover by thermal point bonding, ultrasonic bonding, adhesive bonding, or a combination thereof.

18. The disposable absorbent article of claim 17, wherein the adhesive bonding may be applied by any pattern of at least one adhesive bead, line, swirl, dot, or combination thereof independently in the machine direction, cross machine direction, or a combination thereof.

19. The disposable absorbent article of claim 18, wherein the at least one adhesive bead is less than about 0.25 inches in width.

20. The disposable absorbent article of claim 1, wherein the outer cover provides an active landing material.

21. The disposable absorbent article of claim 1, wherein the outer cover comprises at least one attachment panel.

22. The disposable absorbent article of claim 21, wherein the at least one attachment panel comprises loop material.

23. A disposable absorbent article comprising:
(i) an outer cover
(ii) at least one single piece of flexible material with an active fastening surface;
(iii) a first waist region;
(iv) a second waist region opposite the first waist region wherein:
(a) the active fastening surface of the at least one single piece of flexible material provides an at least one fastener; and
(b) the outer cover defines an inside surface and an outside surface and includes a pair of ears in the first waist region; such that in use, the active fastening surface of the at least one fastener anchors to the outside surface of the ears of the outer cover in the first waist region and the at least one fastener fastens the outside surface of the outer cover in the second waist region thereby forming a waist opening and a pair of leg openings.

24. The disposable absorbent article of claim 23, wherein the pair of ears comprise an elastomeric material.

25. The disposable absorbent article of claim 24, wherein the elastomeric material is a neck-bonded laminate (NBL).

26. The disposable absorbent article of claim 23, wherein the at least one fastener comprises a anchor end and a user end.

27. The disposable absorbent article of claim 26, wherein the anchor end of the at least one fastener is reinforced with glue.

28. The disposable absorbent article of claim 26, wherein the anchor end of the at least one fastener has a shear strength of greater than 3,000 grams tensile.

29. The disposable absorbent article of claim 26, wherein the anchor end of the at least one fastener has a length of about 0.75 to about 1.25 inches and a width of about 1.5 to about 4.0 inches.

30. The disposable absorbent article of claim 26, wherein the user end of the at least one fastener has a shear strength of greater than 2,000 grams tensile.

31. The disposable absorbent article of claim 26, wherein the user end of the at least one fastener has a width of about 0.75 to about 2.0 inches.

32. The disposable absorbent article of claim 23, which is an incontinence product.

33. The disposable absorbent article of claim 23, which is a diaper.

34. The disposable absorbent article of claim 23, wherein the active fastening surface of the at least one single piece of flexible material is substantially covered by active fastening material.

35. The disposable absorbent article of claim 34, wherein the active fastening material comprises a plurality of hooks.

36. A disposable absorbent article comprising:
(i) an outer cover
(ii) at least one single piece of flexible material with an active fastening surface;
(iii) a first waist region;
(iv) a second waist region opposite the first waist region wherein:
(a) the active fastening surface of the at least one single piece of flexible material provides an at least one fastener; and
(b) the outer cover defines an inside surface and an outside surface; such that in use, the active fastening surface of the at least one fastener anchors to the outside surface of the outer cover in the first waist region and is affixed to the outside surface of the outer cover in the first waist region with adhesive bonding; and the at least one fastener fastens the outside surface of the outer cover in the second waist region thereby forming a waist opening and a pair of leg openings.

37. The disposable absorbent article of claim 36, which is an incontinence product.

38. The disposable absorbent article of claim 36, which is a diaper.

39. The disposable absorbent article of claim 36, wherein the active fastening surface of the at least one single piece of flexible material is substantially covered by active fastening material.

40. The disposable absorbent article of claim 39, wherein the active fastening material comprises a plurality of hooks.

41. The disposable absorbent article of claim 36, wherein the outer cover comprises a pair of ears.

42. The disposable absorbent article of claim 41, wherein the pair of ears comprise an elastomeric material.

43. The disposable absorbent article of claim 42, wherein the elastomeric material is a neck-bonded laminate (NBL).

44. The disposable absorbent article of claim 36, wherein the at least one fastener comprises a anchor end and a user end.

45. The disposable absorbent article of claim 44, wherein the anchor end of the at least one fastener is affixed to the at least one outside surface of the outer cover by thermal point bonding, ultrasonic bonding, adhesive bonding, or a combination thereof.

46. The disposable absorbent article of claim 45, wherein the adhesive bonding may be applied by any pattern of at least one adhesive bead, line, swirl, dot, or combination thereof independently in the machine direction, cross machine direction, or a combination thereof.

47. The disposable absorbent article of claim 46, wherein the at least one adhesive bead is less than about 0.25 inches in width.

48. The disposable absorbent article of claim 44, wherein the anchor end of the at least one fastener has a shear strength of greater than 3,000 grams tensile.

49. The disposable absorbent article of claim 41, wherein the anchor end of the at least one fastener has a length of about 0.75 to about 1.25 inches and a width of about 1.5 to about 4.0 inches.

50. The disposable absorbent article of claim 44, wherein the user end of the at least one fastener has a shear strength of greater than 2,000 grams tensile.

51. The disposable absorbent article of claim 44, wherein the user end of the at least one fastener has a width of about 0.75 to about 2.0 inches.
